(19) United States
(12) Patent Application Publication
(10) Pub. No.: US 2009/0069748 A1
(43) Pub. Date: Mar. 12, 2009

(54) PUSHABLE BALLOON CATHETER ASSEMBLY

(75) Inventor: DARIN G. SCHAEFFER, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, Bloomington, IN (US)

(21) Appl. No.: 11/854,150

(22) Filed: Sep. 12, 2007

(51) Int. Cl. A61M 25/10; A61M 25/09; B28B 21/42

(52) U.S. Cl. 604/103.09; 264/635; 604/164.13; 604/96.01

(57) ABSTRACT

A pushable balloon catheter assembly for use in a body vessel. The catheter includes an outer shaft having an outer wall defining an outer lumen and an inner shaft having an inner wall disposed within the outer lumen. An inflatable balloon having a balloon wall defining a balloon interior is attached at a distal tip of the assembly. The balloon wall defines a proximal aperture cooperable with a distal portion of the outer shaft at a common axial location of the catheter assembly. At least part of the outer wall is attached to at least part of the inner wall at the common axial location to define an inflation orifice in fluid communication between the outer lumen and a balloon interior for inflation of the balloon for treatment of the body vessel.

### BACKGROUND OF THE INVENTION

1. Field of the Invention

The present invention generally relates to medical catheters. More specifically, the invention relates pushable balloon catheters.

2. Description of Related Art

Pushable balloon catheters are used for the treatment of many conditions relating to body vessels including arteries and veins. For such treatments a wire guide may be percutaneously inserted into the body vessel and positioned near a location where treatment is necessary. The catheter may be inserted over the wire guide and the distal tip of the catheter guided to the treatment location along the wire guide. Once at the treatment location, a balloon at a distal tip of the catheter is unfolded and inflated to, for example, temporarily occlude the body vessel. In some examples, a stent may be disposed around the balloon in which case inflation of the balloon positions and expands the stent within the body vessel. In other examples, the wire guide may be removed and another treatment device inserted in its place. Once the wire guide is removed and the balloon is unfolded, or the stent is positioned, existing catheters have a tendency to undesirably give or bend adjacent the distal tip.

In view of the above, it is apparent that there exists a need for an improved pushable balloon catheter.

### SUMMARY OF THE INVENTION

In satisfying the above need, as well as overcoming the enumerated drawbacks and other limitations of the related art, the present invention provides a pushable balloon catheter assembly for use in a body vessel. The catheter assembly includes an outer shaft having a distal portion and an outer wall defining an outer lumen. An inner shaft is disposed within the outer lumen and includes a distal end and an inner wall defining an inner lumen. An inflatable balloon has a balloon wall defining a balloon interior, a proximal aperture, and a distal aperture. The proximal aperture is cooperable with the distal portion of the outer wall at a common axial location and the outer wall is attached to at least part of the inner wall at the common axial location, thereby providing additional axial stiffness. In some examples, at least part of the inner shaft may be disposed within the balloon interior, and the distal end may be cooperable with the distal aperture of the balloon. An inflation orifice is in fluid communication between the outer lumen and the balloon interior is defined between the inner and outer walls for inflation of the balloon.

In one embodiment, the inflation orifice is provided between the outer wall and the inner wall and defines an approximately circular shape. In other examples, the closed figure is arcuate in shape including, for example, a crescent shape.

In another embodiment, the proximal aperture of the balloon, the outer wall and the inner wall are heat bonded together and in still another embodiment, they are attached together using an adhesive.

Another example of the present invention includes providing heat shrink tubing over the proximal aperture and the outer wall at the common axial location. One example of heat shrink tubing includes fluorinated ethylene-propylene tubing.

The present invention also includes a catheterization apparatus including an introducer sheath having a sheath wall defining a sheath lumen. Any of the pushable balloon catheter assemblies described herein may be disposed within the sheath lumen of the introducer sheath and a wire guide may be positioned within the inner lumen of the catheter assembly. Both the pushable balloon catheter and the wire guide are configured for relative axial movement within their respective lumens. The wire guide of the present assembly is made of any appropriate material including stainless steel and shape memory materials including, for example, Nickel-Titanium alloys.

A method of making a pushable balloon catheter assembly is also encompassed by the present invention. The method includes disposing an inner shaft having an inner wall defining an inner lumen within an outer lumen defined by an outer wall of an outer shaft. A proximal aperture of an inflatable balloon is positioned over a distal portion of the outer shaft. An inner building mandrel is inserted within the inner lumen of the inner shaft, and an outer building mandrel is inserted between the outer wall and the inner wall within the outer lumen. The proximal aperture and at least part of the inner wall are attached to the distal portion before removing the inner and outer building mandrels.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a side view of one embodiment of a catheterization apparatus for use with a pushable balloon catheter assembly according to the present invention;

FIG. 2 is an exploded view of the catheterization apparatus of FIG. 1;

FIG. 3 is a sectional view of the distal tip of the catheter assembly of FIG. 2 along the line 3-3;

FIG. 4A is a sectional view through one embodiment of the catheter assembly of FIG. 2 along a line 4-4 adjacent a distal tip;

FIG. 4B is a sectional view of another embodiment of the catheter assembly shown in FIG. 4A;

FIG. 5 is a sectional view of a distal tip of the catheter assembly of FIG. 2 along a line 3-3 during fabrication of the catheter assembly; and

FIG. 6 is a flow-chart describing a method of making the catheter assembly according to the present invention.

### DETAILED DESCRIPTION

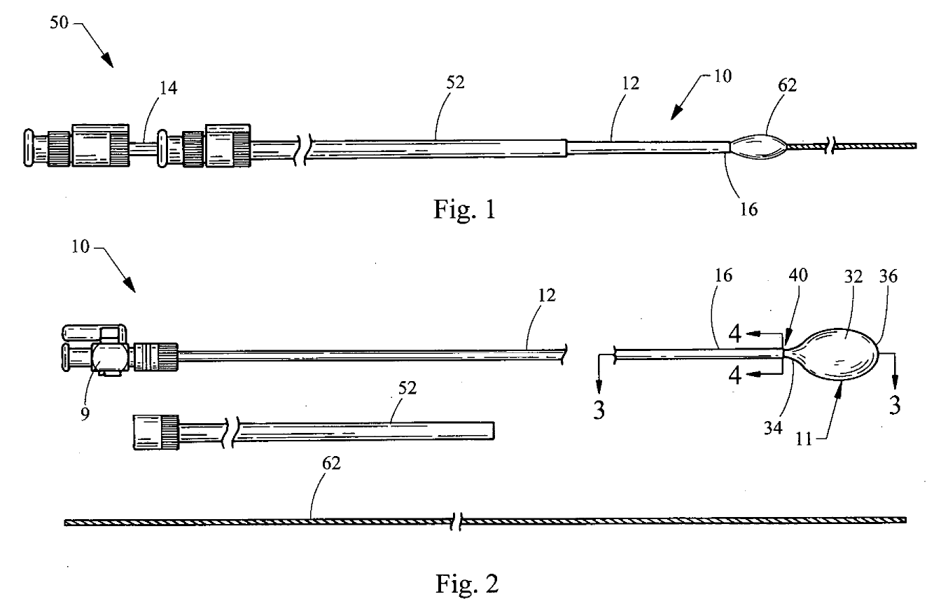

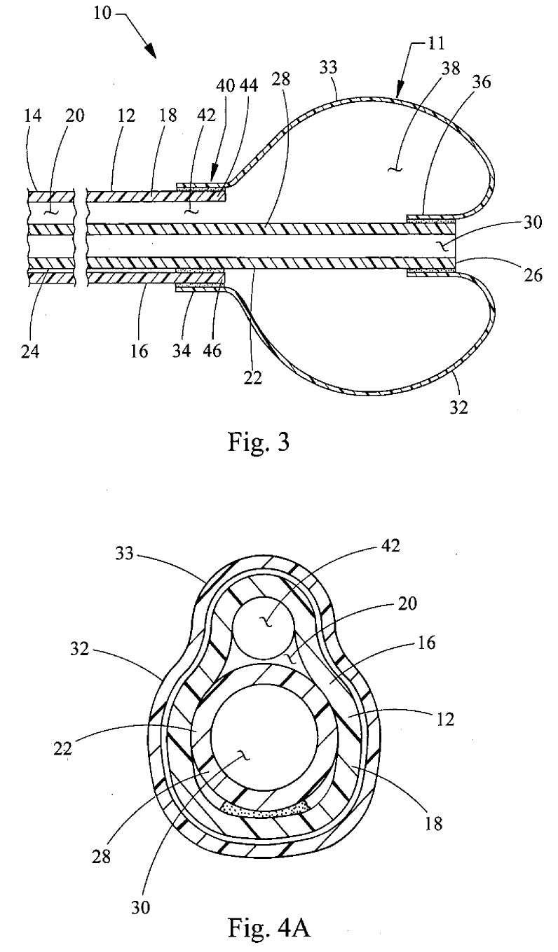

Referring now to FIGS. 1 and 2, a pushable balloon catheter assembly embodying the principles of the present invention is illustrated therein and designated at 10. The assembly 10 extends from a proximal hub 9 to a distal tip 11. As its primary components, the assembly 10 includes an outer shaft 12 extending from a proximal portion 14 to a distal portion 16. An inner shaft 22, best shown in FIG. 3, extends from a proximal end 24 to a distal end 26 and is disposed within the outer shaft 12. An inflatable balloon 32 is cooperable with the distal portion 16 of the outer shaft 12.

The outer shaft, inner shaft and inflatable balloon are made of any appropriate flexible material for use as a catheter in a body vessel. The material may include, for example, nylon, polyester, polytetrafluroethylene (PTFE), latex, rubber and mixtures thereof.

Turning now to FIG. 3, the distal tip 11 of the catheter assembly 10 is shown in more detail. The outer shaft 12 has an outer wall 18 defining an outer lumen 20. The inner shaft 22 is disposed within the outer lumen 20, and has an inner wall 28 defining an inner lumen 30. The inflatable balloon 32 has a balloon wall 33 defining a balloon interior 38, a proximal aperture 34, and a distal aperture 36. The distal aperture 36 is cooperable with the distal end 26 of the inner shaft 22 such that at least part of the inner shaft 22 is disposed within the balloon interior 38. The proximal aperture 34, the outer shaft 12, and a part of the inner shaft 22 are all cooperable and attached together at a common axial location 40 of the distal portion 16 of the outer shaft 12.

Attaching the above features together at the common axial location 40 increases the stiffness of the distal tip 11 such that it is less likely to give or bend when the catheter assembly 10 is pushed longitudinally in a distal direction after, for example, the balloon 32 has been inflated or otherwise unfolded, or another treatment device, such as a stent, has been deployed. The above features may be attached together, for example, by at least one of an appropriate heat bonding technique, adhesive, or a combination thereof. While the balloon wall 33 is shown as a separate element attached to the inner and outer shafts 12 and 22, in some examples it may be possible for all three to be integrally formed without falling beyond the scope of the present invention. In other examples, heat shrink tubing (not shown) may optionally be disposed over the proximal aperture 34 of the balloon 32 and the outer wall 18. The heat shrink tubing may be any appropriate material including, but not limited to, fluorinated ethylene-propylene tubing.

An inflation orifice 42 is provided in fluid communication between the outer lumen 20 and the balloon interior 38 at the common axial location 40. In the example shown, the inflation orifice 42 is preferably positioned between the outer wall 18 and the inner wall 28. The inflation orifice 42 allows, for example, fluid injected into the proximal portion 14 of the outer lumen 20 to enter and inflate the balloon 32 for treatment of the body vessel. As a result, at one circumferential location 44, adjacent to the inflation orifice 42, only two layers (i.e. the balloon wall 33 and the outer wall 18) are attached together. At another circumferential location 46, away from the inflation orifice 42, all three layers are attached together.

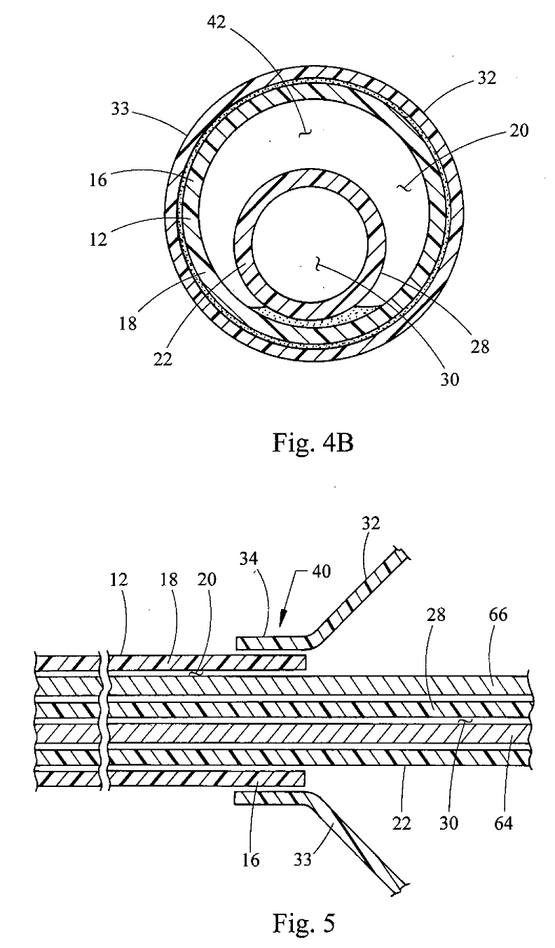

As best shown in FIGS. 4A and 4B, the inflation orifice 42 may have various appropriate cross-sectional shapes. In the example of FIG. 4A, the orifice 42 is substantially circular. In the example of FIG. 4B, the orifice 42 has an arcuate or crescent shape. It should be noted that the particular shape of the orifice 42 is determined by the shape of mandrels used during the manufacturing process to fabricate the catheter assembly 10 (see below).

Returning back to FIGS. 1 and 2, the catheter assembly 10 is shown, for example, as part of a catheterization apparatus 50 for treatment of the body vessel in accordance with one embodiment of the present invention. As shown, the delivery assembly 50 includes a PTFE introducer sheath 52 for percutaneously introducing the catheter assembly 10 into a body vessel. Of course, any other suitable material for the introducer sheath 52 may be used without falling beyond the scope or spirit of the present invention. The introducer sheath 52 may have any suitable size, for example, between about three-french to eight-french. The introducer sheath 52 serves to allow the catheter assembly 10 to be percutaneously inserted to a desired location in the body vessel. The introducer sheath 52 receives and provides stability to the catheter assembly 10 at a desired location of the body vessel. For example, the introducer sheath 52 is held stationary within a common visceral artery, and adds stability to the catheter assembly 10 it is advanced through the introducer sheath 52 to a desired treatment location in the vasculature.

The apparatus 50 may also include a wire guide 62 configured to be percutaneously inserted within the vasculature to guide the catheter assembly 10 to the desired location. The inner lumen 30 is fed over the wire guide 62 to provide the catheter assembly 10 with a path to follow as it is advanced within the body vessel. The size of the wire guide 62 is based on an inside diameter of the inner lumen 30 of the catheter assembly 10 and the diameter of the target body vessel.

When the distal tip 11 of the catheter assembly 10 is at the desired location in the body vessel, the wire guide 62 may optionally be removed and another treatment device, for example, an embolic protection device (not shown) releasably coupled to a stylet. The stylet and embolic protection device may be inserted into the proximal end 24 of the inner shaft 22 of the catheter assembly 10. The stylet may be advanced through the inner shaft 22 for deployment of the device through the distal tip 11 to, for example, capture emboli in the body vessel during treatment of a lesion. The hub 9 is configured to receive the treatment device for advancement through the inner lumen 30. The size of the catheter assembly 10 is based on the size of the body vessel in which it percutaneously inserts, and the size of any additional devices.

This embodiment may also be used to retrieve devices by positioning the distal tip 11 of the catheter assembly 10 adjacent a deployed device in the vasculature (not shown). An inner member or stylet is advanced through the inner lumen 30 of the catheter assembly 10 until it protrudes from the distal tip 11. A distal segment of the stylet may then be coupled to a retrieval end of the device and the stylet is retracted proximally, drawing the device into the inner lumen 30.

It is understood that the apparatus described above is merely one example of an apparatus that may employ the catheter assembly 10 described herein for treatment of a body vessel. Of course, other apparatus, assemblies and systems may be used to deploy any embodiment of the catheter assembly 10 without falling beyond the scope or spirit of the present invention.

As best shown in FIG. 5, the catheter assembly 10 is fabricated by first disposing the inner shaft 22 within the outer lumen 20 and positioning the proximal aperture 34 of the balloon 32 over the distal portion 16 of the outer shaft 12. An inner building mandrel 64 is inserted within the inner lumen 30 of the inner shaft 22 and an outer building mandrel 66 is inserted between the outer wall 18 and the inner wall 28. The proximal aperture 34 and a portion of the inner wall 28 are both attached to the distal portion 16 of the outer wall 18 at the common axial location 40. In this example, the inner mandrel 64 is circular in cross-section with a diameter substantially the same as a diameter of the inner lumen 30 to maintain the existing shape of the inner lumen 30, while in other examples the inner mandrel 64 may have other shapes. Likewise, the outer mandrel 64 determines the shape of the inflation orifice 42.

Returning to FIGS. 4A and 4B, the inflation orifice 42 may have any appropriate cross-sectional shape determined by the outer mandrel 64 as noted above. In some examples to simplify manufacturing, the outer mandrel 64 may have a circular cross-section. However, heat bonding the catheter assembly 10 has the potential to reduce the area of the orifice 42 since the outer wall 18 is subject to conforming at least partially to the shape of the inner and outer mandrels 64 and 66 and the inner shaft 22 as shown in FIG. 4A. To maximize the area of the orifice 42, the outer mandrel 64 may have a non-circular, arcuate or crescent cross-section (see FIG. 4B). Preferably, an inside and outside radii of the crescent cross-section will substantially match radii of the respective inner and outer walls 28 and 18.

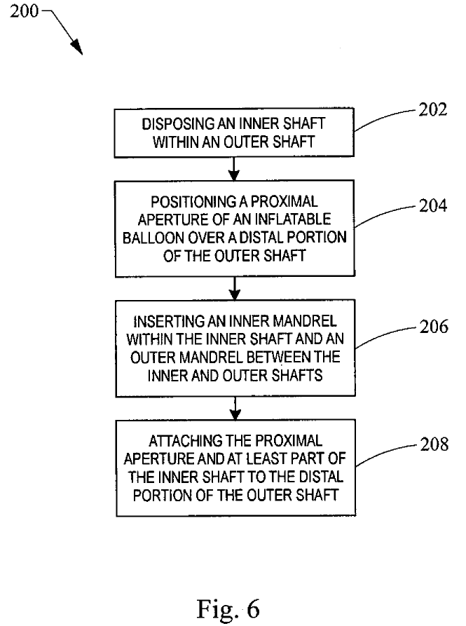

Turning to FIG. 6, it provides a flow chart designated at 200 describing one exemplary method for making the catheter assembly 10. The method includes, at box 202, disposing an inner tubular shaft within an outer tubular shaft. Box 204 includes positioning a balloon wall of an inflatable balloon having a proximal aperture over a distal portion of the outer shaft. Box 206 includes inserting an inner mandrel within the inner shaft and an outer mandrel between the inner and outer shafts. Box 208 includes attaching the balloon wall at least part of the inner shaft to the distal portion of the outer shaft at a common axial location

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

I claim:

1. A pushable balloon catheter assembly having an axial length for use in a body vessel, the catheter assembly comprising:
an outer shaft having a proximal portion extending to a distal portion and an outer wall defining an outer lumen formed therethough;
an inner shaft being disposed within the outer lumen of the outer shaft, the inner shaft having a proximal end extending to a distal end and including an inner wall defining an inner lumen formed therethrough; and
an inflatable balloon having a balloon wall defining a balloon interior, the wall having a proximal aperture cooperable with the distal portion of the outer wall at a common axial location with the outer wall, the outer wall being attached to at least part of the inner wall at the common axial location to define an inflation orifice in fluid communication between the outer lumen and the balloon interior for inflation of the balloon to treat the body vessel.

2. The catheter assembly according to claim 1, wherein the balloon wall has a distal aperture, the balloon wall extending from the proximal aperture to the distal aperture, the distal aperture being cooperable with the distal end of the inner shaft.

3. The catheter assembly according to claim 1, wherein at least part of the inner shaft is disposed within the balloon interior.

4. The catheter assembly according to claim 1, wherein the inflation orifice is formed between the outer wall and the inner wall.

5. The catheter assembly according to claim 1, wherein the inflation orifice has a circular cross-section.

6. The catheter assembly according to claim 1, wherein the inflation orifice has a crescent cross-section.

7. The catheter assembly according to claim 1, wherein the proximal aperture of the balloon, the outer wall and the inner wall are attached together by at least one of heat bonding, and an adhesive.

8. The catheter assembly according to claim 1, wherein the outer shaft, inner shaft and inflatable balloon are made of at least one of nylon, polyester, polytetrafluroethylene, latex, and rubber.

9. The catheter assembly according to claim 1, further comprising heat shrink tubing being disposed over the proximal aperture and the outer wall at the common axial location.

10. The catheter assembly according to claim 9, wherein the heat shrink tubing includes fluorinated ethylene-propylene tubing.

11. A catheterization apparatus for use in a body vessel, the assembly comprising:
an introducer sheath extending from a proximal section to a distal section and having a sheath wall defining a sheath lumen extending therethrough;
a pushable balloon catheter assembly having an axial length being disposed within the sheath lumen of the introducer sheath for relative axial movement therein, the catheter assembly comprising:
an outer shaft having a proximal portion extending to a distal portion and an outer wall defining an outer lumen formed therethough;
an inner shaft being disposed within the outer lumen of the outer shaft, the inner shaft having a proximal end extending to a distal end and including an inner wall defining an inner lumen formed therethrough;
an inflatable balloon having a balloon wall defining a balloon interior, the wall having a proximal aperture and a distal aperture and extending therebetween, the distal aperture being cooperable with the distal end of the inner shaft, and the proximal aperture being cooperable with the distal portion of the outer wall at a common axial location with the outer wall, the outer wall being attached to at least part of the inner wall at the common axial location to define an inflation orifice in fluid communication between the outer lumen and the balloon interior for inflation of the balloon; and
a wire guide including a distal part being disposed within the inner lumen of the inner shaft for relative axial movement therein, the distal part being positioned adjacent a treatment location within the body vessel to guide the balloon catheter to the treatment location.

12. The catheterization apparatus according to claim 11, wherein at least part of the inner shaft is disposed within the balloon interior.

13. The catheterization apparatus according to claim 11, wherein the inflation orifice is provided between the outer wall and the inner wall.

14. The catheterization apparatus according to claim 11, wherein the inflation orifice is a circle.

15. The catheterization apparatus according to claim 11, wherein the inflation orifice is a crescent shape.

16. The catheterization apparatus according to claim 11, wherein the proximal aperture of the balloon, the outer wall and the inner wall are attached together by at least one of heat bonding, and an adhesive.

17. The catheterization apparatus according to claim 11, further comprising heat shrink tubing being disposed over the proximal aperture and the outer wall at the common axial location.

18. The catheterization apparatus according to claim 11, wherein the wire guide is made of a shape memory material including Ni—Ti.

19. A method of making a pushable balloon catheter assembly, the method comprising:
disposing an inner shaft having an inner wall defining an inner lumen within an outer lumen defined by an outer wall of an outer shaft;
positioning a proximal aperture of an inflatable balloon over a distal portion of the outer shaft;
inserting an inner building mandrel within the inner lumen of the inner shaft, and inserting an outer building mandrel between the outer wall and the inner wall within the outer lumen;
attaching the proximal aperture and at least part of the inner wall to the distal portion; and
removing the inner and outer building mandrels.

20. The method according to claim 19, further comprising:
positioning a distal aperture of the inflatable balloon over a distal end of the inner shaft; and
attaching the distal aperture to the distal end.
